(19) United States
(12) Patent Application Publication
(10) Pub. No.: US 2003/0072726 A1
(43) Pub. Date: Apr. 17, 2003

(54) Skin lightening agents

(76) Inventors: Nigel E. Banister, London (GB); Peter S.J. Cheetham, Kent (GB)

Correspondence Address: ROSENTHAL & OSHA L.L.P., 1221 MCKINNEY AVENUE, SUITE 2800, HOUSTON, TX, 77010, US

(21) Appl. No.: 09/928,878

(22) Filed: Aug. 13, 2001

Related U.S. Application Data
(63) Continuation of application No. PCT/GBOO/00490, filed on Feb. 14, 2000.

(30) Foreign Application Priority Data
Feb. 13, 1999 (GB) GB 9903216.1

(51) Int. Cl. A61K 7/135

(52) U.S. Cl. 424/62

(57) ABSTRACT

Compounds having formula (I) may be used as skin lightening agents wherein R¹ is CH═CH₂, COOH or CH₂—CH₃; R³ is OH, or —OCH₃; R⁴ is OH; and R², R⁵ and R⁶ are all hydrogen, the compound of formula (I) optionally being in the form of a salt, when it contains a COOH and/or OH group.

The present invention relates to compounds from natural biological materials such as plant material which display unexpectedly good skin lightening properties and their use as skin lightening agents.

There are three main uses for skin lighteners; to lighten age spots (liver spots or senile lentigo), to reduce the brown-black colour of non-Caucasian skin and to prevent the darkening of Caucasian and Japanese skins. The main mechanism of action is believed to involve inhibition of the enzyme tyrosinase that is involved in the metabolism of tyrosine into melanin in the melanocytes present in the skin. Therefore, to be effective, it is desirable that a skin lightener should inhibit mammalian tyrosinase effectively, should be permeable through both the cell and melanocyte membranes and should be safe and non-toxic to skin cells at the concentration it is to be used at.

Commonly used skin lighteners are hydroquinone, arbutin and kojic acid. Hydroquinone is the most effective but has significant adverse effects so that it is no longer registered for use in the EC. Many other materials have also been used or documented as skin lighteners, including lactic acid, ferulic acid and nicotinamide as well as plant extracts such as Bearberry extract. Other skin lighteners include glycosides and esters of hydroxysalicylic acid, ascorbyl methylsilanol and liquorice extracts.

The use of caffeic acid or an ester or amide thereof to depigment the skin is described in U.S. Pat. No. 5,164,185. Depigmenting compositions containing di- or tri- caffeoylquinic acid are disclosed in U.S. Pat. No. 5,445,816.

The naturally occurring compound ferulic acid is known to have skin lightening activity. Ferulic acid is a phenylpropenyl molecule.

According to the invention, there is provided the use of a compound having the following formula I in the manufacture of a composition for use as a skin lightening agent:

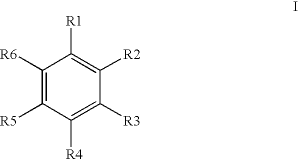

wherein

R¹is CH═CH₂, COOH or CH₂—CH₃;

R³ is OH, or —OCH₃;

R⁴ is OH; and

R², R⁵and R⁶ are all hydrogen,

the compound of formula I optionally being in the form of a salt (including mono- , di- or poly-valent salts), when it contains a COOH and/or OH group.

Preferably in the compounds of formula I, R¹ is CH═CH₂, COOH or CH₂—CH₃.

The compound of formula I is preferably selected from vinylguaiacol, protocatechuic acid and ethylguaiacol. More preferably, the compound is vinylguaiacol or protocatechuic acid, most preferably vinylguaiacol.

By “skin lightener” we include the meaning that the compound inhibits melanogenesis, especially the enzymes (such as tyrosinase) which are associated with the formation of melanin by mammalian cells, such as melanocytes in the skin.

The compounds of the invention may have one or more other useful activities selected from antioxidant, antimicrobial, antibrowning, aroma/flavour and acidulant activities. Skilled persons will appreciate that the antibrowning and skin lightening activities of the compounds of the invention are linked in that, without intending to be bound in any way by scientific theory, they appear to work by inhibiting enzymes associated with coloured material formation. For example, the compounds inhibit polyphenol oxidase (tyrosinase and laccase) enzymes which catalyse formation of brown-black pigments (melanoids) formed by oxidative polymerization of plant phenols such as chlorogenic acid in plant tissue. Similarly, the compounds of the invention inhibit tyrosinase enzyme in the skin to prevent melanin formation from precursor materials present in skin cells, such as tyrosine.

The compounds of the invention can be used in a wide range of applications because of their versatility. A particular advantage of the compounds of the invention is that they display solubility in both oil and water for ease of formulation

It has surprisingly been found that the compounds of the invention exhibit paricularly effective skin lightening activity when they are used together with another skin lightening agent. For example, glycosides such as arbutin exhibits a synergistic skin lightening effect when used together with protocatechuic acid.

A particularly advantageous feature of the compounds of the invention is that they may possess, in addition to their skin lightening activity, other different activities ie, they have multiple functionality eg, they may also have acidulant, flavour/aroma, antioxidant, antimicrobial or antibrowning activity. Hence, a single compound of the invention may replace a plurality of compounds in known skin lightening compositions.

A still further advantage of the compounds of the invention is that they may possess a pleasant aroma and this may avoid the need for the separate addition of a perfume in various compositions and formulations.

Compounds such as vinylguaiacol and ethylguaiacol possess both a pleasant aroma and antimicrobial activity, making them especially suitable for use in formulations for application to the skin.

Vinylguaiacol and ethylguaiacol both have a fresh camphoraceous/herbal/medicated character bringing to mind some pharmaceutical cough preparations. Thus, they convey hygiene, cleanliness and well being.

The compounds may be prepared by a bioprocess which comprises treating a substrate with one or more microorganisms selected from Rhodotorula, Saccharomyces (eg, S. cerevisiae), Paecilomyces, Candida and Paenibacillus; wherein the substrate is selected from ferulic acid or caffeic acid.

An advantageous feature of such bioprocesses is that they are natural, that is, they involve biological, especially enzymatic, processes and the molecules are readily biodegradable because they occur in nature and, indeed, are already present in the human food supply from vegetable, food and beverage sources.

Skilled persons will appreciate that the bioprocesses are not limited to the specific examples, but include micro-organisms and/or enzymic and/or cell free extracts and/or engineered cells or enzymes therefrom which exhibit a suitable enzymic activity.

The micro-organism or enzyme or cell-free extract derived therefrom may produce the desired product efficiently and in high yields. This may be quantified in terms of: the rate of production of the product (gl⁻¹day ⁻¹); the concentration of the product that accumulates (gl⁻¹); the yield of the product obtained from the substrate (g of product per g of substrate or % M yield); and the absence of side products which is reflected in the purity of the isolated product (% purity).

The strains may exhibit tolerance to high concentrations of both the substrate and the product, for example at least 1 gl⁻¹, preferably in the range of 1 to 40 gl⁻¹, more preferably in the range of 5 to 40 gl⁻¹. The strains may also exhibit high metabolic selectivity for the production of the required products, for example the products may be produced in at least 75% reaction molar yield and at least 50% recovered molar yield, and they have the ability to produce the products as non-growing cells so that, for example, expensive nutrients do not have to be supplied and expensive sterile fermentation equipment does not have to be used.

In particular, the criteria for establishing suitability of the micro-organism or enzyme or cell-free extract for use in the methods of the invention are as follows:

The micro-organism or enzyme or cell-free extract derived therefrom may produce at least 1 g of the desired product per liter of reaction fluid and/or at least 50% molar yield of the desired product from the substrate (eg ferulic acid or caffeic acid) at a concentration of >0.5 gl⁻¹. The desired product may have a purity of at least 90% as determined by positive characterisation of the product by ab initio analytical methods such as NMR.

The micro-organism or enzyme or cell-free extract may be capable of being used repeatedly, in two phase reaction systems, as immobilised cells, as disrupted cells, and is capable of reacting with impure substrates, especially plant extracts, if required.

Preferably, the bioprocess includes a biphasic reaction mixture. More preferably, the biphasic reaction mixture includes an aqueous phase, such as water, and a water immiscible (eg, organic liquid) phase such as vegetable oil, for example miglyol. The water immiscible phase acts as a product ‘sink’ in which the desired product formed from the substrate accumulates. This prevents accumulation of the product in the aqueous phase to levels which may inhibit or terminate the enzymatic reaction.

This results in increased product yields compared to when the bioprocess is performed using a monophasic reaction mixture.

The product should be produced over a reasonably short period of time eg 1 to 3 days or less. Preferably, the test microorganism is isolated using the soil isolation protocol described hereinafter.

Preferably, one or more of the compounds of the invention are provided in the form of an extract from a plant material. Suitable extracts from plant materials include, for example, a maize extract containing vinylguaiacol (eg, in an amount of greater than 50%) and an onion skin extract containing protocatechuic acid. The onion skin extract has unexpectedly good skin lightening properties and is a preferred aspect of the invention.

In another aspect, the invention provides compositions comprising a compound having a formula I as defined previously in combination with another skin lightening agent.

The other skin lightening agent is preferably selected from: ascorbic acid, Kojic acid, hydroquinone and arbutin and plant extracts containing them such as from bearberry.

The invention further provides a composition comprising a combination of two or more different compounds having the formula I.

In the compositions of the invention, the compound of formula I, or, where the compositions contain two or more compounds of formula I, at least one of the compounds, may be in encapsulated form.

Preferred embodiments of the invention will now be described with reference to the following illustrative examples.

Bioprocess for Making Compounds of Formula I

In the following examples, analysis of vinylguaiacol and ethylguaiacol was carried out using high performance liquid chromatography (hplc) using the following conditions:

| | |
|---|---|
| Column | Spherisorb C-18 |
| Mobile phase | 60:40 deionised water: |
| | MeCN; 1% acetic acid |
| Flow rate | 2 mlmin⁻¹ |
| Detection | Ultraviolet at 290 nm. |

Analysis of protocatechuic acid was carried out using high performance liquid chromatography (hplc) using the following conditions:

| | |
|---|---|
| Column | Spherisorb C-18 |
| Mobile phase | 80:20 deionised water: |
| | MeCN; 1% acetic acid |
| Flow rate | 1.75 mlmin⁻¹ |
| Detection | Ultraviolet at 290 nm. |

In the following examples, where organisms are grown in culture broth, the growth medium can contain specified amounts of either, or both, a vitamin supplement and a trace elements supplement. These were prepared as follows.

Vitamin supplement: biotin (2 mgl⁻¹), folic acid (2 mgl⁻¹), pyridoxine (10 mgl⁻¹), riboflavin (5 mgl⁻¹), thiamine (5 mgl⁻¹), nicotinic acid (5 mgl⁻¹), pantothenic acid (5 mgl⁻¹), vitamin B12 (0.1 mgl⁻¹), 4-aminobenzoic acid (5 mgl⁻¹), and thioacetic acid (5 mgl⁻¹).

Trace elements supplement: concentrated hydrochloric acid (51.3 mll⁻¹), MgO (10.75 gl⁻¹), CaCO₃ (2.0 gl⁻¹), FeSO₄.7H₂O (4.5 gl⁻¹), ZnSO₄.7H₂O (1.44 gl⁻¹), MnSO₄.4H₂O (1.12 gl⁻¹), CuSO₄.5H₂O (0.25 gl⁻¹), CoSO₄.7H₂O (0.28 gl⁻¹), and H₃BO₃ (0.06 gl⁻¹).

Commercial supplies of Sacharomyces cerevisiae from Tesco plc, Sainsburys plc or Hovis yeast were used in Examples 2 and 8.

All other organisms were isolated using the soil isolation protocol described, unless indicated otherwise.

| | Identification | | |
|---|---|---|---|
| Patent | of micro- | | |
| Example | organism | Organism characteristics | Where isolated |
| 1 | Rhodotorula | Yeast with orange, mucoid | From air onto a |
| | glutinis | colonies. Colony form: | yeast malt agar |
| | IMI 379894 | circular, entire margin | plate. |
| | | convex elevation. | |
| 3 | See example | See example 1 | |
| | 1 | | |
| 4 | See example | See example 1 | |
| | 1 | | |
| 5 | Paecilo- | Filamentous fungus | From fruiting |
| | myces | producing light brown | body of a fungus |
| | variotii | powdery spores | growing in |
| | IMI 379901 | | Amazon in Brazil |
| 6 | Candida | | |
| | versitalis | | |
| | NCYC 1433 | | |
| 8 | Candida | | |
| | versitalis | | |
| | NCYC 1433 | | |
| 10, 11 | Paeni- | Gram positive, group 2 | Bird nesting site |
| | bacillus | bacillus with oval, centrally | at Dorking, UK |
| | polymyxa | positioned endospore and a | |
| | IMI 382464 | thick, ridged coat | |

Abbreviations
IMI—International Mycological Institute, Egham, Surrey, UK
NCYC—National collection of Yeast Cultures, Norwich, UK

Soil Isolation Protocol

To 2 ml deionised water was added approximately 100 mg soil The resulting suspension was mixed thoroughly (vortex mixer) allowed to stand at room temperature (22° C. for 1 hour followed by further mixing to distribute suspended material. The macroscopic solids were allowed to settle for approximately 10 minutes and the supernatant (100 μl) was applied to a suitable medium (see below) in a 90 mm petri dish using a spread plate technique. Plates were incubated at 28° C. until colony development was observed.

For the isolation of fungi, soil supernants were spread plated onto a yeast malt medium comprising: 4 g glucose, 4 g yeast extracts, 10 g malt extract per liter deionised water.

For the isolation of bacteria, soil supernatants were spread plated onto nutrient agar (Oxoid, Unipath Limited, UK)

### EXAMPLE 1

### Preparation of Vinylguaiacol

A strain of Rhodotorula glutinis (IMI 379894) was cultured at 30° C. by shaking at 200 rpm on a yeast malt medium containing (per liter of deionised water): glucose 4 g; yeast extract 4 g and malt extract 10 g. After 40 hours incubation, ferulic acid was added to a final concentration of 2 gl⁻¹. The incubation was continued for a further 21 hours during which time the progress of the reaction was monitored by h.p.l.c. analysis using the conditions described above.

After 21 hours incubation the reaction had progressed to a molar conversion of 97.4%. The molar conversion after 3 hours was 61%.

### EXAMPLE 2

### Preparation of Vinylguaiacol

Saccharomyces cerevisiae,“Bakers yeast”, (2 g, purchased from J. Sainsburys plc under the trade mark Sainsburys Easy Blend) was activated by suspending dry yeast powder in deionised water (20 ml) for 30 minutes at 37° C. A medium (1 l) containing (per liter deionised water): glucose 4 g; yeast extract 4 g and malt extract 10 g was inoculated (5%) with the activated yeast suspension and incubated at 30° C. with shaking at 200 rpm for 96 hours. After 96 hours incubation, the cells were harvested by centrifugation (15 minutes at 4000 rpm), resuspended in 50 ml of 0.9% (w/v) NaCl, and then disrupted by passage once through a cell disrupter (operating pressure 30,000 psi). To 50 ml of the resultant disrupted cell suspension was added ferulic acid at an initial concentration of 10 gl⁻¹. Also added at the same time was 50 ml of Miglyol to form an upper organic layer to the biphasic biotransformation mixture. The progress of the reaction was monitored by analysis as described above.

After incubation at 30° C. for 64 hours the reaction had progressed to a 92% conversion to vinylguaiacol.

### EXAMPLE 3

### Preparation of Vinylguaiacol From Maize Fibre

Ferulic acid was released from maize fibre as follows. A 10 g portion of maize fibre was shaken (200 rpm) at 30° C., overnight, in a conical flask with 100 ml of 1M sodium hydroxide solution. The resulting solution was acidified to pH 5.5 prior to the addition of 45 ml of a culture of Rhodotorula glutinis (IMI 379894) which had been grown on yeast malt medium in a 250 ml shake flask and incubated with shaking (200 rpm) at 30° C. for 40 hours. At this time a concentration of 0.495 gl⁻¹ ferulic acid was detected. The resulting suspension was itself incubated at 30° C. with shaking (200 rpm) and the vinylguaiacol concentration monitored by hplc. After 10 minutes a 7.9% conversion of ferulic acid to vinylguaiacol was observed; after 1 hour there was a 29% conversion; after 20 hours a 93% conversion. The reaction mixture was extracted twice with 50 ml of n-hexane and the combined extracts dried and evaporated to yield 48 mg of an oil comprising 84% vinylguaiacol.

### EXAMPLE 4

### Preparation of Vinylguaiacol From Maize Fibre

Ferulic acid was released from maize fiber as follows. A 50 g portion of maize fibre was shaken (200 rpm) at 30° C., for 15 hours, in a conical flask with 500 ml of IM sodium hydroxide solution. The resulting solution containing 940 mg ferulic acid was neutralised by the addition of concentrated hydrochloric acid. This was added to 1 liter of a culture of Rhodotorula glutinis (IMI 379894) which had been grown on yeast malt medium in a 5 liter shake flask and incubated with shaking (200 rpm) at 30° C. for 24 hours. The mixture was adjusted to pH 5.5 and 1 liter of n-hexane was added. The resulting two-phase system was mixed gently (80 rpm) at 30° C. After 24 hours the two liquid phases were separated and the aqueous re-extracted with 500 ml of n-hexane. The combined organic solvent phases, which contained 540 mg vinylguaiacol (75% yield) were dried and evaporated to yield an oil (740 mg) which was 65% vinylguaiacol by assay. This represents a 66% recovery of vinylguaiacol from ferulic acid.

### EXAMPLE 5

### Preparation of Protocatechuic Acid From Caffeic Acid

To 400 ml of sterilised yeast malt medium (4 g glucose; 4 g yeast extract; 10 g malt extract; made up to 1 liter with deionised water) was added glucose (40 g) and caffeic acid (1 g) and the resultant mixture was inoculated with spores of Paecilomyces variotii (IMI 379901) prior to incubation at 30° C. with shaking at 200 rpm. Further aliquots of glucose (20 g) were added at 24 hours, 72 hours and 96 hours. After 168 hours, hplc assay indicated that there were 630 mg total of protocatechuic acid present in the reaction system, representing a 74% molar conversion. The culture broth was extracted with ethyl acetate (900 ml) and assay showed that 527 mg of protocatechuic acid had been recovered along with 45 mg of unreacted caffeic acid. Evaporation of the dried solvent yielded 750 mg of a pale yellow gum which was resuspended in diethyl ether (100 ml) to give a red, granular, insoluble solid which was removed and the remaining solution evaporated to give 700 mg of recovered solid which was 67% protocatechuic acid by assay and 6% caffeic acid. This solid was dissolved in diethyl ether (10 ml) to which was then added a further 10 ml of petroleum ether 40/60. Evaporation of this solution by blowing nitrogen over the solution gave a yellow oil from which the solution was decanted and evaporated to give a cream coloured solid (435 mg) which was 96.3% protocatechuic acid by assay.

### EXAMPLE 6

### Production of Ethylguaiacol From Ferulic Acid Using C. versitalis

Candida versitalis (NCYC 1433) was grown from a plate culture inoculum for 6 days in yeast malt medium containing 10 g/L malt extract, 4 g/L yeast extract, 4 g/L glucose, and 2% sodium chloride dissolved in deionised water and autoclaved at 120° C. The 50 ml culture was incubated at 30° C. and 200 rpm in a 250 ml conical flask.

After 6 days this culture was used to provide a 10% inoculum for a 150 ml second culture of the yeast malt medium occupying 50% v/v of the flask. This was incubated at 21-22° C. for 24 hrs while agitating at 150 rpm. Then ferulic acid was added to a concentration of 2 g/L, together with 100 ml of Miglyol, which alternatively could be added after 50 hrs when the concentration of ethylguaiacol in the aqueous phase had reached 0.25-0.3 g/L. (Miglyol is added because the strain appears to be intolerant of the ethylguaiacol product, with the maximum concentration of ethylguaiacol accumulated (in a monophasic reaction) in the absence of Miglyol as product sink being 0.5 g/L).

Ethylguaiacol formation was monitored by hplc using as solvent 60:40 water:acetonitrile plus, 1% acetic acid, at a flow rate of 2 ml/min and monitoring at 290 nm. Ethylguaiacol was formed in a good yield from ferulic acid, with vinylguaiacol being detected as the intermediate. After 184 hrs incubation, the concentration of ethylguaiacol in the Miglyol was 3.64 g/L, which represents 92 to 94% of the theoretical maximum yield. The ethylguaiacol could be easily recovered from the Miglyol as a pure chemical by solvent extraction into hexane and then rotary evaporation to dryness.

### EXAMPLE 7

### Reduction of Vinylguaiacol to Ethylguaiacol

Vinylguaiacol was produced from ferulic acid by microbial bioconversion as described in Example 1 or Example 2.

Vinylguaiacol (500 mg) and cobalt (II) sulfate heptahydrate (940 mg) were dissolved in ethanol (10 ml) under a helium atmosphere. Sodium borohydride (255 mg), dissolved in ethanol (5 ml), was added slowly with stirring in an ice bath. The dark solution was then permitted to stir at room temperature. After 36 hours an hplc trace showed that only about 4% of the starting material remained. A peak with the same retention time as ethylguaiacol (6.0 mins, 60:40 H₂O: MeCN+1% AcOH) was present. The ethanolic solution was poured into 2M HCl (20 ml) and extracted twice with diethyl ether. The organic layer was dried over sodium sulfate. Ethanol (100 ml) was added to the diethyl ether and the latter was removed in vacuo to leave an ethanolic solution. Ethylguaiacol was recovered in a yield of 72% from the starting ferulic acid, and with no residual vinylguaiacol remaining in this recovered ethylguaiacol product.

### EXAMPLE 8

### Production of Vinylguaiacol in the Presence of Miglyol

Candida versitalis (Zyl 866; NCYC 1433) was grown (from a 10% inoculum) in 25 ml of sterile yeast malt medium containing 2% w/v sodium chloride. After 24 hours ferulic acid was added to a final concentration of 4 g/l and 25 ml of miglyol was also added as a supernatant. The flask was shaken at 250 rpm at 21° C. and the solutions assayed by HPLC.

Over 134 hours the amount of vinylguaiacol increased in the miglyol phase of the reaction with relatively little vinylguaiacol being present in the aqueous phase of the bioconversion. There was also evidence of ethylguaiacol being present in the miglyol phase of the reaction. By comparison of peak areas on HPLC it was seen that use of 8 g/l ferulic acid in the reaction led to similar amounts of vinylguaiacol being produced whereas 2 g/l or 1 g/l ferulic acid in the reaction gave lower quantities of vinylguaiacol.

### EXAMPLE 9

### Extraction of Protocatechuic Acid From Onion Skins

Chopped onion waste material (200 g dry weight) was suspended in 0.1 M NaOH (1 l). This 20% w/v suspension was heated at 90° C. for 4 hours in a water bath. The suspension was then pressed to remove the solid material and the solids were washed with sufficient deionised water to return the volume of the liquor to 1 l. The liquor contained 0.7 g/l protocatechuic acid (PCA); therefore the yield as a percentage of the dry material used was 0.35% w/w. The liquor was re-heated to 90° C. Sodium hydroxide (10 M) was added to a final concentration of 0.1 M, then chopped waste onion material (200 g) was suspended in the liquor, and this suspension was again heated at 90° C. for 4 hours. The suspension was pressed and the solids were washed as before. The 1 l of liquor contained 1.17 g/l PCA. The overall release efficiency in terms of dry weight yield was therefore 0.29%. This reloading was repeated two more times to achieve a final PCA concentration of 2.76 g/l in the liquor corresponding to an overall yield of 0.32% w/w of dry onion material added.

Extraction of PCA From Onion Skin Liquor

Reloaded onion skin liquor containing 2.3 g/l PCA (1 l total, 2.30 g PCA), was adjusted to pH 3 with concentrated HCl and centrifuged at 4000 rpm for 20 minutes. The resultant supernatant totalled 910 ml and contained 2.35 g/l PCA (2.14 g PCA). The clarified aqueous layer was extracted with an equal volume of n-butyl acetate. After 24 hours the aqueous layer contained 0.48 g/l PCA (20% of the original concentration). Therefore the organic layer contained 1.84 g/l PCA (910 ml total, 1.68 g PCA). The solvent was then removed in vacuo to leave a solid (2.75 g) which was shown to be 60% PCA (by HPLC).

TABLE 1
Preparation of compounds of the invention from caffeic acid or ferulic acid and selected microorganisms.

| | Reaction molar | | | Productivity | | |
|---|---|---|---|---|---|---|
| | yield % (g | | | Biocatalyst (g | Approx. yield | |
| | substrate | Product g 1⁻¹ | Recovered | product g cells ⁻¹ | from 5001 | |
| Product | supplied 1⁻¹) | culture | molar yield (%) | dry wt.) | fermenter (kg) | Zyl No. |
| Ferulic acid to | 94 (8.5) | 6.1 | 87 | 0.95 | 2.4 | 702 |
| vinylguaiacol | | | | | | |
| Caffeic acid to | 51 (4) | 1.75 | n.d. | n.d. | 0.88 | 733 |
| Protocatechuic acid | | | | | | |
| Ferulic acid to | 92-94 | 3.64 | n.d. | n.d. | 0.73 | NCYC 1433 |
| ethylguaiacol | | | | | | |

### EXAMPLE 10

### Preparation of Vinylguaiacol From Ferulic Acid Using Paenibacillus polymyxa

Paenibacillus polymyxa (Zyl 277; IMI CC Deposit No 382464) cells were grown at 30° C., shaking at 200 rpm for 27 hours on a medium comprising per liter deionised water: (NH₄)₂ SO₄, 5 g; K₂HPO₄, 2 g; NaCl, 0.2 g; glucose, 10 g; malt extract, 3 g; yeast extract, 3 g; MgSO₄, 0.22 g; CaCl₂, 0.015 g; ferulic acid, 0.5 g. The cells were harvested by centrifugation (4,000×g 15 m′) washed with 0.9% (w/v) saline solution followed by resuspension in 0.9%(w/v) saline solution as a 20-fold concentration. An aliquot of concentrated cells (5 ml) was added to a solution of sodium alginate (15 ml 3.5% w/v) and mixed thoroughly, prior to addition dropwise from a 3 ml plastic pipette into 1 liter of 0.2 M CaCl₂ solution. The beads formed by this procedure were stored at 4° C. overnight in CaCl₂ solution to harden before washed in 21 of tap water.

The beads interspersed with an inert packing material were packed into a 100 ml glass column. A solution of ferulic acid in tap water (500 ml, 6 g/l) was pumped continuously through the column at a temperature of 24° C. and the pH of this solution was maintained at pH 7.0. After 6 hours operation, the aqueous stream exiting the top of the column was continuously extracted into hexane (500 ml) to remove vinylguaiacol, prior to returning to the column.

Results

Vinylguaiacol concentrations (gl⁻¹) were:

| Time (Hr) | Aqueous | Hexane | Total |
|---|---|---|---|
| 1.75 | 0.074 | — | 0.074 |
| 3 | 0.116 | — | 0.116 |
| 6 | 0.175 | — | 0.175 |
| 23 | 0.101 | 0.8 | 0.901 |
| 48 | 0.144 | 1.332 | 1.476 |
| 122 | 0.152 | 2.09 | 2.24 |

### EXAMPLE 11

### Production of Vinylguaiacol from Ferulic Acid by Paenibacillus polymyxa (ZYL277) in a Two Phase System

Paenibacillus polymyxa (Zyl 277; IMI CC Deposit No 382464) was grown in a bioreactor in a medium containing (g/l) (NH₄)₂SO₄, 5; K₂HPO₄, 2; NaCl, 0.2; yeast extract, 2; malt extract, 2; glucose, 10; ferulic acid, 0.5; 10 ml/l of a solution containing 0.1 M MgSO₄/0.01 M CaCl₂; at 30° C., pH 6.0, oxygen 70% on a stirrer cascade (100-500 rpm).

After 24 h, 100 ml of culture was placed in a 250 ml conical flask, stirred at 25° C. with pH control at 7 using 2 M NaOH or dilute phosphoric acid as required. 4 g/l ferulic acid (free acid) was added to the aqueous phase before it was overlaid with 100 ml hexane. The hexane was added to partition vinylguaiacol from the aqueous phase where it may be toxic to the organism. Vinylguaiacol concentrations in both the aqueous and hexane phase were determined by HPLC. Further ferulic acid was added to the aqueous phase as the reaction proceeded. The hexane layer was removed periodically and replaced with 100 ml of new hexane to prevent it becoming saturated with vinylguaiacol. Vinylguaiacol concentrations in both phases at the time of changing the hexane phase are shown below along with the cumulative total ferulic acid added to the aqueous phase (g/l).

| | | Vinylguaiacol (g/l) | | Total Ferulic Acid |
|---|---|---|---|---|
| Time (h) | Aqueous | Hexane | Total | Added (g/l) |
| 21 | 0.70 | 9.78 | 10.48 | 12 |
| 93 | 0.78 | 9.86 | 10.64 | 22 |
| 117 | 0.70 | 10.44 | 11.14 | 34 |
| 143 | 0.60 | 7.94 | 8.54 | 42 |
| 172 | 0.37 | 7.30 | 7.67 | 50 |
| 262 | 0.62 | 10.58 | 11.20 | 58 |
| 314 | 0.53 | 9.40 | 9.93 | 66 |

The seven collected hexane layers contained a total of 5.42 g vinylguaiacol. Ferulic acid additions had been carried out to take account of increases in aqueous volume due to pH control. In total 7.16 g ferulic acid had been added (equivalent to 66 g/l taking account of increasing aqueous volume). This equates to a molar yield for vinylguaiacol of 98%.

### EXAMPLE 12

### Skin Lightener Assay Method

Tyrosine: To 171.4μl of a solution of tyrosine (1.5 mM) in phosphate buffer (100 mM, pH6.4) was added of an inhibitor solution (100 μg ml⁻¹) in phsophate buffer (100 mM, pH6.4) in a quartz cuvette. The reaction mixture was made up to 980 μl with phosphate buffer (100 mM, pH6.4) and the reaction initiated by the addition of tyrosinase (Sigma, 220 μl, 1100 units/ml in phosphate buffer (100 mM, pH6.4)), The reaction was monitored by the increase in absorbance at 470 nm over 10 minutes.

TABLE 2
In vitro Skin lightening Data

| | Degree of Inhibition |
|---|---|
| Tyrosmase Inhibitor | IC₅₀* (μg/ml) |
| Vinylguaiacol | 21.5 |
| Ethylguaiacol | 40 |
| Hydroquinone+ | 2.27 |
| Bearberry extract+ | 3.1 |
| Bearberry extract+ | 4.4 |
| Kojic acid+ | 6.5 |

*Concentration of inhibitor required reducing tyrosinase activity to 50%
+Standards

Melanogenesis Inhibition (Skin Lightening) Assays

These were conducted using cultured melanocyte cells exposed to dilutions of the best materials, and compared with kojic acid, a brown skin lightener. The degree of melanin production was inversely proportional to the skin lightener activity. Any cytotoxic effect of the test materials is reflected in a decrease in melanin formation.

Controls

Each 96 well assay plate included a solvent control and a blank control. Eight concentrations of kojic acid were used as a positive control.

Subculturing the Cells into 96 Well Plates

When the cell cultures were 50 to 80% confluent, the growth medium was removed from the flasks and the cultures rinsed with 10 ml of HBSS. 2 ml of trypsin/EDTA solution was added and the flasks were incubated at 37° C.+/−1° C. for 2 to 5 minutes. When the cells started to dislodge, the flask was rapped sharply against the palm of the hand and approximately 5 ml of assay medium added to neutralise the trypsin.

The concentration of cells was determined by counting an aliquot of the stock cell suspension in a haemocytometer. A seeding suspension of 5×10⁴ cells/ml was prepared in assay medium. One hundred microlitres of the seeding cell suspension was added to the appropriate wells on each 96 well plate. One hundred microlitres of growth medium was added to the outer wells to maintain humidity. The cells were incubated at 37+/−1° C. in a humidified atmosphere containing 5+/−1% CO₂ in air for 24 hours.

Skin Lightening Assay

Based on the information available for the test article, eight decreasing doses were selected and used in the assay. The highest dose of test article was a concentration of 5 mg/ml, based on its solubility in the solvent. In the case of the mixture of protocatechuic acid and arbutin, a 1 to 1 w/w mixture of each was used in each assay with the concentration of each in the highest dose assay being 2.5 mg/ml, and then pro rata in the dilutions assayed in the other 7 assays. The maximum solvent concentration (other than assay medium) was 1%.

The test article and positive control was tested by treating six wells per dilution of B16-F1 cells seeded approximately 24 hours earlier. Prior to treatment the medium was removed, and 200 μl of the pre-warmed (37 +/−1° C.) test article dilutions, positive control dilutions and solvent control, were added to the appropriate wells. All test article concentrations were dosed into a single outer well (200 μl) of the corresponding plate to serve as a turbidity control. The remaining wells around the edge of the plate, designated as blanks, received 200 μl of assay medium prior to incubation. Following dosing, the plates were incubated at 37+/−1° C. for 96 hours +/−2 hours.

After exposure, 150 μl of each treatment solution (containing any secreted melanin) was removed and transferred to the corresponding wells of a new 96 well plate (melanin production plate). Turbidity controls were also transferred. The absorbance of the melanin present in the melanin production plates was measured at 405 nm (OD₄₀₅) with an Anthos 2010™microplate reader.

The cells remaining in the assay plate were used to determine the cytotoxicity of the test article (cytotoxicity plate). The remaining media was removed and 100 μl of growth medium containing 25 μg/ml of neutral red was added to each well. The plates were returned to the incubator for 3 hours +/−5 minutes, after which time the NR medium was decanted and the cells washed with 150 μl of PBS. The PBS was removed by gently tapping the plate. One hundred and fifty μl of NR desorb solution was added to each well. After a minimum of 20 minutes incubation at room temperature, the plates were agitated and the absorbance of the neutral red was measured at 540 nm (OD₅₄₀) with an Anthos 2010™ microplate reader.

Melanin Production

The mean of the six OD₄₀₅ values for each concentration of test article and positive control was calculated. The turbidity control values were subtracted to obtain a correct OD₄₀₅ value. The mean of the six OD₄₀₅ values for the corresponding negative or solvent control were calculated by subtracting the blank control value. The corrected OD₄₀₅ values of the test article or positive control was divided by the corrected OD₄₀₅ value for the negative or solvent control to obtain a melanin production ratio.

Cytotoxicity

The mean of the six OD₄₀₅ values for each concentration of test article and positive control was calculated. The blank control value was subtracted to obtain a corrected OD₄₀₅ value. The mean of the six OD₅₄₀ values for the corresponding negative or solvent control was calculated in the same way. The corrected OD₅₄₀ values of the test article or positive control was divided by the corrected OD₅₄₀ value for the negative or solvent control to obtain a cytotoxicity ratio.

The results are given in Table 4.

TABLE 3
Solubilities, taste, odour and colour of compounds of the invention

| | Solubility (g/l) | | | | | | Colour |
|---|---|---|---|---|---|---|---|
| | Water | | | Ethylene | Propylene | Taste/ | (Solution in |
| Molecule | (pH 7.0) | Ethanol | Oil | Clycol | Clycol | Odour¹ | Ethanol¹) |
| Vinylguaiacol | >2 | >100 | >50 | — | — | Cloves | Colourless |
| Protocatechuic acid | >300 | >500 | <0.2 | 250 | — | None | Colourless |
| Ethylguaiacol | ND | ND | ND | 250 | — | Smoky | Colourless |

¹Determined at effective concentrations
ND Not Determined

Melanogenesis Inhibition Activities
(at concentrations of 0.015-15 mg/ml)

| | 0.015 | 0.05 | 0.15 | 0.5 | 1.5 | 5 | 15 |
|---|---|---|---|---|---|---|---|
| Protocatechuic acid | 0.5 | 2 | 1 | 3 | 7 | 0 | 0 |
| Vinylguaiacol | 3 | 6 | 10 | 1 | 0 | 0 | 0 |
| Ethylguaiacol | 2 | 8 | 14 | 17 | 0 | 0 | 0 |
| Onion skin extract | 2 | 2 | 7 | 25 | 88 | 0 | 0 |
| (Example 9) | | | | | | | |
| Arbutin* | 0 | 1 | 4 | 11 | 17 | 14 | 11 |
| Kojic acid* | 0 | 0 | 0 | 2 | 57 | 170 | 255 |
| Protocatechuic acid plus | 4 | 15 | 2 | | 52 | 0 | 0 |
| arbutin (50/50 mixture) | | | | | | | |

*Comparative examples

For the purposes of the invention “cosmetic products” are products intended for increasing the appeal, visually and olfactively, of, the human body. Likewise “personal care products” are products intended for cleaning, smoothing or otherwise improving the health and well-being of the outside of the human body. These definitions of cosmetic and personal care products at least partially overlap since many products provide functions in both categories. Examples of such products are: perfumes and like products known as “eau de toilette” and “eau de parfum”, hand and body lotions, skin tonics, shaving products, bath and shower products, deodorant and antiperspirant products, hair care products such as shampoos and hair conditioners, mouth and dental care products. Such products are well known in the art. Thus, examples of skin care products are described in “Harry's Cosmeticology”, R. G. Harry, 6^{th} edition, Leonard Hill Books (1973), Chapters 5-13, 18 and 35; examples of deodorants and antiperspirants are described in C. Fox, cosmetics and Toiletries 100 (Dec. 1985), pp 27-41; examples of hair care products are described “Harry's Cosmeticololgy”, vide supra, chapters 25-27; examples of dental care products are described in M. Pader, Oral Hygiene: Products and Practice, Marcel Dekker, New York (1988). Cosmetic and personal care products are usually perfumed, on the one hand to give pleasant odour to the products themselves and on the other hand to have the body parts to which they are applied emit a pleasant odour after their use.

Tables 4 to 6: Personal Care Products including the compounds of the invention

TABLE 4
A photostable sunscreen lotion

| Ingredient | % w/w |
|---|---|
| Glyceryl monomyristate | 5.00 |
| Cetyl alcohol | 2.0 |
| Butylmethoxy dibenzoylmethane | 1.00 |
| Isopropyl myristate | 7.00 |
| Oleoyl alcohol | 3.00 |
| Polysiloxane A | 5.00 |
| DEA cetylphosphate | 3.00 |
| Water, deionised | 67.00 |
| Propylene glycol | 6.00 |
| A compound of the invention | 0.60 |
| Fragrance | 0.30 |

TABLE 5
All purpose dry skin cream

| Ingredient | % w/w |
|---|---|
| Phase A(80° C.) | |
| Glyceryl stearate SE | 12.00 |
| Stearyl stearate (Hetester 412) | 4.00 |
| Di-C 12-15 Alkyl fumarate (Marrix | 2.00 |
| SF) | |
| Ganex V220 | 2.00 |
| Minno 21 | 6.00 |
| Phase B (80°) | |
| Water, deionised | 63.00 |
| Glycerin | 10.00 |
| Phase C | |
| A compound of the invention | 1.00 |

Add Phase A to Phase B. Mix at 80° C. Cool to 60° C. and add Phase C. Cool to 50° C. and package.

TABLE 6
Moisturiser with sun protection

| Ingredient | % w/w |
|---|---|
| Phase A (45° C.) | |
| Water deionised | 63.40 |
| Phase B (dry blend) | |
| Magnesium aluminium silicate (Veegum Reg.) | 1.00 |
| Xanthan Gum | 0.50 |
| Phase C (45° C. and dissolve) | |
| Propylene glycol isoceteth-3 acetate (Hetester | 10.00 |
| PHA) | |
| Octyl methoxycinnamate | 7.50 |
| Octyldodecyl neopentanoate (Elefac 1-205) | 5.00 |
| Benzophenone-3 | 2.50 |
| Minno 21 | 5.00 |
| Octyl salicylate | 5.00 |
| Phase D | |
| A compound of the invention | 0.10 |

Add phase B to phase A; disperse well and hold at 45° C. Then add phase C slowly. Hold at 45° C., mix well then add phase D. Cool and mix to 30° C. and pack.

### EXAMPLE 13

### Skin Lightener Cream Formulation

A formulation was produced containing a compound of the invention by formulating the following ingredients:

Water, Butylene glycol, Arbutin, PEG 8, Alcohol, Jojoba oil, Squalane, Dimethicone, Glycerin, Petrolatum, Pentaerythrityl tetraoctanoate, PEG-60 hydrogenated castor oil, Behenyl alcohol, Potassium carbomer, Butyl alcohol, Methylparaben, Octyl methoxycinnamate, Benzophenone-3, Compound of formula I, Xanthan gum, Tocopheryl acetate, Stearyl glycyrrhetinate, Panthenenyl ethyl ether, Ethylparaben, Cholesteryl hydroxystearate, Trisodium edta, Sodium hyaluronate, Sodium citrate, Sodium metabisulfite, Tocopherol, Iron oxides.

### EXAMPLE 14

### Skin Whitener

| Ingredients | % weight |
|---|---|
| Surfac GMS SE40 | 10.00 |
| Mineral Oil | 2.00 |
| Waglinol 6014 | 2.00 |
| Cetearyl Alcohol | 2.00 |
| DC 200/350 | 1.00 |
| Nipasol M | 0.10 |
| Eusolex 6007 | 0.50 |
| Water | To 100% |
| Propylene Glycol | 5.00 |
| Empicol LZV | 0.60 |
| Nervanaid BA2 | 0.10 |
| Compound of formula I | 2.00 |
| Ascorbic Acid | 0.10 |
| Sodium Metabisulphite | 0.15 |
| Irgasan DP300 | 0.10 |
| Fragrance | qs |
| Colour | qs |

### Micro-Organism Deposits

Exemplary micro-organisms suitable for use in accordance with the present invention have been deposited for the purposes of patent procedures under the Budapest Treaty with the IMI Genetic Resource Reference Collection which is an International Depositary authority recognised under the Treaty. The address of the IMI Collection is CABI Bioscience UK Centre Egham, Genetic Resource Collection, Bakeham Lane, Egham, Surrey, England TW20 PTY telephone 01784 470111, fax 01491 829100, e-mail bioscience@cabi.org.

| | Strain | Date of acceptance | IMI CC |
|---|---|---|---|
| Micro-organisms | Number | for patent purposes | Number |
| Paecilomyces variotti | ZYL 733 | 20 Nov. 1998 | 379901 |
| Rhodotorula glutinis | ZYL 702 | 20 Nov. 1998 | 379894 |
| Paenibacilus polymyxa | ZYL 277 | 24 Jan. 2000 | 382464 |

TABLE 7
Exemplary compounds of the invention

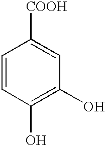

Protocatechuic acid
(3,4-Dihydroxybenzoic acid)

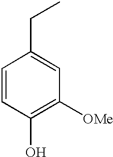

Ethylguaiacol,
(4-Ethyl-2-methoxyphenol)

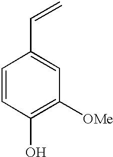

4-Vinylguaiacol
(4-Vinyl-2-methoxyphenol)

1. Use of a compound having the following formula I in the manufacture of a composition for use as a skin lightening agent: 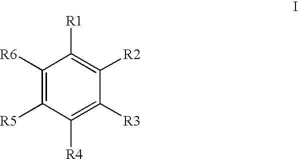
wherein
R¹ is CH═CH₂, or CH₂—CH₃;
R³ is OH, —OCH₃;
R⁴ is OH; and
R², r⁵ and R⁶ are all hydrogen, the compound of formula I optionally being in the form of a salt, when it contains an OH group.

2. Use of a compound having the formula I in the manufacture of a composition for use as a skin lightening agent 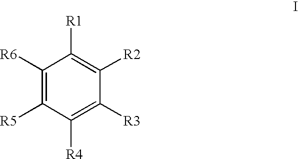
wherein
R¹ is CO₂
R³ is OH
R⁴ is OH
and R², R⁵ and R⁶ are all H, the compound of formula I optionally being in the form of a salt.

3. Use as claimed in claim 1 wherein the compound is selected from vinylguaiacol and ethylguaiacol.

4. Use as claimed in claim 1, wherein the compound is vinylguaiacol.

5. Use as claimed in claim 1, wherein the compound is ethylguaiacol.

6. Use as claimed in claim 2, wherein protocatechuic acid is used together with another skin lightening agent.

7. Use as claimed in claim 6, wherein the skin lightening agent is arbutin.

8. Use of a compound of formula I as defined in claim 1 or claim 2, as a skin lightening agent.

9. Use as claimed in claim 8, wherein the compound is vinylguaiacol, protocatechuic acid or ethylguaiacol.

10. Use as claimed in any one of claims 1 to 9, wherein the compound is present in a composition for use as a cosmetic or a perfume.

11. Use as claimed in any one of claims 1 to 10, wherein the compound is provided in the form of an extract derived from a plant material that has been subsequently treated with a microorganism.

12. A composition comprising a compound as defined in claim 1 or claim 2 in combination with another skin lightening agent.

13. A composition comprising two or more different compounds, as defined in claim 1 or claim 2.

14. A composition as claimed in claim 13 further comprising another skin lightening agent.

15. A composition as claimed in any one of claims 12 to 14, wherein the compound(s) is(are) in encapsulated form.
